(19) United States
(12) Patent Application Publication
(10) Pub. No.: US 2018/0042726 A1
(43) Pub. Date: Feb. 15, 2018

(54) CRANIAL IMPLANT

(71) Applicant: Michael J. Yaremchuk, Lynnfield, MA (US)

(72) Inventor: Michael J. Yaremchuk, Lynnfield, MA (US)

(21) Appl. No.: 15/676,502

(22) Filed: Aug. 14, 2017

Related U.S. Application Data
(63) Continuation-in-part of application No. 29/546,742, filed on Nov. 25, 2015, now Pat. No. D794794.

(51) Int. Cl. A61F 2/28; A61B 17/17

(52) U.S. Cl. CPC A61F 2/2875; A61B 17/1739; A61F 2002/30948

(57) ABSTRACT

A custom cranial implant for use in a cranial opening in a skull of a patient, the implant including a contoured shell configured to mate with the defect edges of the cranial opening; multiple fixation channels formed within the contoured shell, such that each channel extends from an exterior surface of the contoured shell into a peripheral edge; and one or more drain ports formed within the contoured shell, as well as a kit including the custom cranial implant, a method of manufacturing the implant, and a method of surgically implanting the implant.

### CROSS-REFERENCE TO RELATED APPLICATIONS

This application is a continuation-in-part and claims priority to U.S. Design Patent Application Number 29/546,742, titled “Cranial Implant,” filed Nov. 25, 2015, which is incorporated in its entirety herein by reference.

### FIELD OF THE INVENTION

The present invention relates to cranial implants and, more specifically, to a custom cranial implant having a contoured shell, a drain opening, and multiple oblique fixation channels extending from proximate a peripheral edge on an exterior surface of the contoured shell into a peripheral edge of the contoured shell for attaching the implant to the skull of the patient.

### BACKGROUND OF THE INVENTION

Intracranial neurosurgical procedures typically require craniotomy and removal of a segment of the skull to access the brain. Similarly, massive hemorrhage or swelling of the brain often may mandate removal of portions of the skull to decompress the brain. Accidental trauma to the skull also may result in fracture to the calvarium. Fracture pieces may be lost or deemed too contaminated to be replaced. In each instance, full-thickness bone loss of the cranium may result in exposure of the intracranial contents to the environment making the brain susceptible to injury.

At the completion of the brain surgery or other procedure, the removed bone segment (referred to as a “bone flap”) must be replaced. In instances in which the bone flap is unavailable for replacement, a cranial implant is employed. Thus, cranial implants are conventionally used to replace bone missing from the cranial vault, protecting the brain from various external forces.

Cranial implants typically are constructed of various biocompatible materials including metals and polymer plastics. The implants may be designed and constructed at the time of surgery. In the alternative, implants may also be prefabricated using computer-aided design and computer-aided manufacture (CAD/CAM) techniques. To effect this, data from a high resolution computed tomographic (CT) scan are typically used to create an implant of dimensions and contour specific for the defect being treated. Indeed, CAD/CAM cranial implants are traditionally made to fill exactly a skeletal defect in the cranial vault.

As described in U.S. patent application Ser. No. 13/532,283, entitled “Craniofacial Implant Registration Features and Methods,” to the instant inventor, which was published as U.S. Patent Application Publication Number 2012/0330427 on Dec. 27, 2012 and which is incorporated herein in its entirety by reference, the position of the cranial implant, conventionally, may be stabilized with plates and screws. For example, as shown in FIG. 1, a cranial implant 58 may include a cranial body portion 60 adapted to fill substantially the cranial defect in combination with a cranial thin edge portion 62 disposed along at least a portion of the periphery of the body portion 60.

In the embodiment shown in FIG. 1, a cranial thin edge portion 62 extending over the adjacent intact skull of the patient is formed on a custom CT-generated cranial implant 58. A plurality of openings or apertures, configured to receive fasteners (e.g., screws or lags screws) to attach the cranial implant 58 to the cranium, may be located about the periphery of the cranial thin edge portion 62. Typically, the cranial thin edge portion 62 includes a lateral or radial extent of up to about 1 to 5 mm (or more depending on the location of the scalp incision to the underlying area of skull reconstruction) and a thickness tapering from up to about 1.5 mm (or more) to less than about 1 mm.

Openings or apertures, e.g., for fixation devices, within the thin, tapered cranial thin edge portion 62, typically, are vertical or substantially vertical, i.e., normal, with respect to the exterior surface of the cranial implant 58. Moreover, forming a countersink in the opening or aperture is uncommon. Indeed, providing a countersink in the relatively thin cranial thin edge portion 62 may further weaken an already fragile area.

Complications associated with the surgery and/or the cranial implant, however, may result after placement of cranial implants. Treatment of these complications usually requires implant removal. A contaminated collection of fluid between the brain and the implant is a frequent etiology necessitating implant removal.

For example, it is not uncommon for brain volume to decrease significantly after, for example, trauma or a neurosurgical intervention for a tumor. This volume decrease often results in creation of a space between the surface of the brain and the inner surface of a cranial implant. Fluid susceptible to contamination and infection inevitably collects in this space. Conventional surgical maneuvers to suspend the dura to the inner surface of an implant or employment of implant design that includes an inner surface to fill this space, however, are imperfect. Moreover, conventional implants limit the ability to drain fluid from this space making contamination and infection necessitating therapeutic implant removal more likely.

Another etiology necessitating implant removal is exposure of the implant as a result of prominent fixation hardware eroding through the overlying scalp, potentially leading to implant exposure and/or surgical failure. For example, cranial implants are typically fixed to the adjacent intact cranium, using, for example, miniaturized plates and screws. Typically, these metal implants have a profile above the surface of the implant and the cranium. The scalp overlying the implant and the fixation devices are often compromised by trauma, necessitating neurosurgery, nearby or even overlying surgical incisions and scarring, and particularly, when radiation therapy has been employed. It is not uncommon for the scalp overlying fixation hardware to erode and, consequently, expose the hardware and attached implant. Contamination of the implant ensues necessitating implant removal for treatment.

### SUMMARY OF THE INVENTION

Accordingly, it would be advantageous to provide a cranial implant that overcomes the deficiencies noted in the current state-of-the art.

In a first aspect, some embodiments of the present invention relate to a custom cranial implant adapted to be implanted into a cranial opening in a skull of a patient. In some embodiments, the cranial implant includes a contoured shell configured to mate with the cranial opening and having a peripheral edge (e.g., a surface that is configured to mate with defect edges of the cranial opening); multiple fixation channels formed within the contoured shell, such that each fixation channel extends from an exterior surface of the contoured shell into the peripheral edge; and a drain port(s) formed within the contoured shell for receiving at least one of a drain and a conduit. In some variations, at least one of the fixation channels includes a countersink feature and/or each fixation channel is dimensioned to receive a fixation device to attach the contoured shell to the skull of the patient. Advantageously, a head portion of each fixation device may be contained within the countersink feature of a corresponding fixation channel, such that the head portion does not protrude above the exterior surface of the contoured shell.

In a second aspect, some embodiments of the present invention relate to a method of manufacturing a custom cranial implant for use in a cranial opening in a skull of a patient, using a three-dimensional image of the cranial opening and surrounding tissue. In some embodiments, the method includes forming an implant/skull interface about a peripheral edge of the cranial implant; determining fixation device locations proximate the peripheral edge of the implant; forming an oblique channel at each fixation device location, such that each oblique fixation channel extends from an exterior surface of the contoured shell into the peripheral edge; and forming a drain port(s) through the cranial implant. In some implementations, forming the implant/skull interface includes forming, using the three-dimensional image, the implant/skull interface about the peripheral edge of the cranial implant to mate with defect edges of the cranial opening. In some variations, the method includes forming a cranial contour about the exterior surface of the cranial implant.

In some implementations, the method may include one or more of: adapting the oblique fixation channels to allow corresponding fixation devices to pass obliquely through the implant into cranial bone in the skull, adapting the oblique fixation channels to allow corresponding fixation devices to purchase cranial bone of the skull, forming a countersink feature in each oblique fixation channel and/or determining a fixation device dimension(s) for securing the fixation device to the skull. Advantageously, each countersink feature may be configured to contain a head portion of a fixation device, such that the head portion does not protrude above the exterior surface of the cranial implant.

In a third aspect, embodiments of the present invention relate to a method of surgically implanting a custom cranial implant having a contoured shell into a cranial opening in a skull of a patient. In some applications, the method includes providing a custom cranial implant having at least one drain port, mating the computer designed peripheral edge to corresponding defect edges of the cranial opening and attaching the cranial implant to the skull of the patient. In some implementations, providing a custom cranial implant includes using data from a three-dimensional image of the cranial opening to from the custom cranial implant, using data from a three-dimensional image of the cranial opening to locate a plurality of fixation device locations proximate the peripheral edge of the implant, forming an oblique fixation channel at each fixation device location, such that each oblique fixation channel extends from an exterior surface of the contoured shell into the peripheral edge, and forming a drain port extending from the exterior surface of the contoured shell to an interior surface of the contoured shell. In some variations, a countersink feature may be formed in each oblique fixation channel, each countersink feature configured to contain a head portion of a fixation device, such that the head portion does not protrude above the exterior surface of the contoured shell.

In some implementations, the method may include one or more of: inserting computer designed fixation devices into corresponding oblique channels; inserting each fixation device, such that each fixation devices passes obliquely through the implant into cranial bone in the skull; and/or inserting each fixation device into corresponding oblique fixation channels, such that each fixation device purchases cranial bone of the skull of the patient. In some variations, the method further includes inserting a drain and/or a conduit into each drain port.

In a fourth aspect, embodiments of the present invention relate to a kit for implanting an object into a cranial opening in a skull of a patient. In some embodiments, the kit includes a custom cranial implant and at least one of a drain and a conduit. In some variations, the cranial implant may include a contoured shell configured to mate with the cranial opening and having a peripheral edge (e.g., a surface that is configured to mate with defect edges of the cranial opening); multiple (e.g., oblique) fixation channels formed within the contoured shell, each fixation channel extending from an exterior surface of the contoured shell into the peripheral edge; and at least one drain port formed within the contoured shell. In some variations, the kit may further include multiple fixation devices.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a side perspective view of a cross-section of a cranial implant in accordance with the prior art;

FIG. 2 is a top perspective view of a cranial implant in accordance with some embodiments of the present invention;

FIG. 3A is a top view of the cranial implant of FIG. 2;

FIG. 3B is left side view of cross-section 3B-3B in FIG. 3A;

FIG. 4 is a cranial implant attached to the patient's skull in accordance with some embodiments of the present invention;

FIG. 5 is an illustration of a contoured shell mated with a cranial opening of a patient in accordance with some embodiments of the present invention;

FIG. 6 is an illustration of the contoured shell of FIG. 5 attached to the patient's skull at the cranial opening in accordance with some embodiments of the present invention; and

FIG. 7 is a flowchart of a method of manufacturing a custom cranial implant in accordance with some embodiments of the present invention.

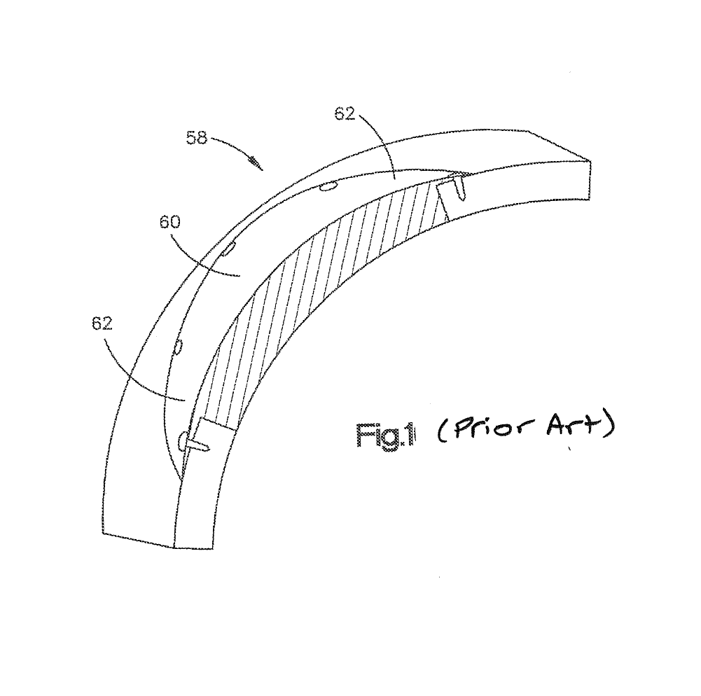

### DETAILED DESCRIPTION

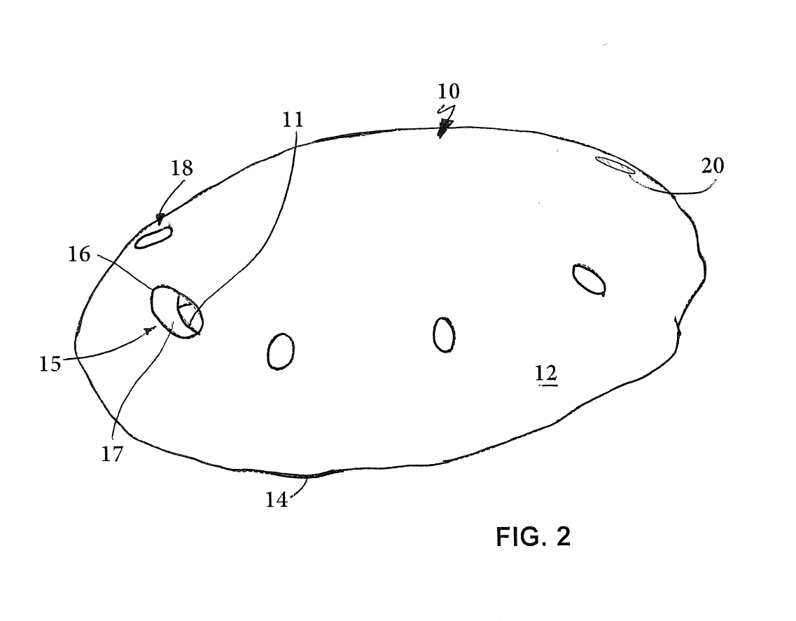

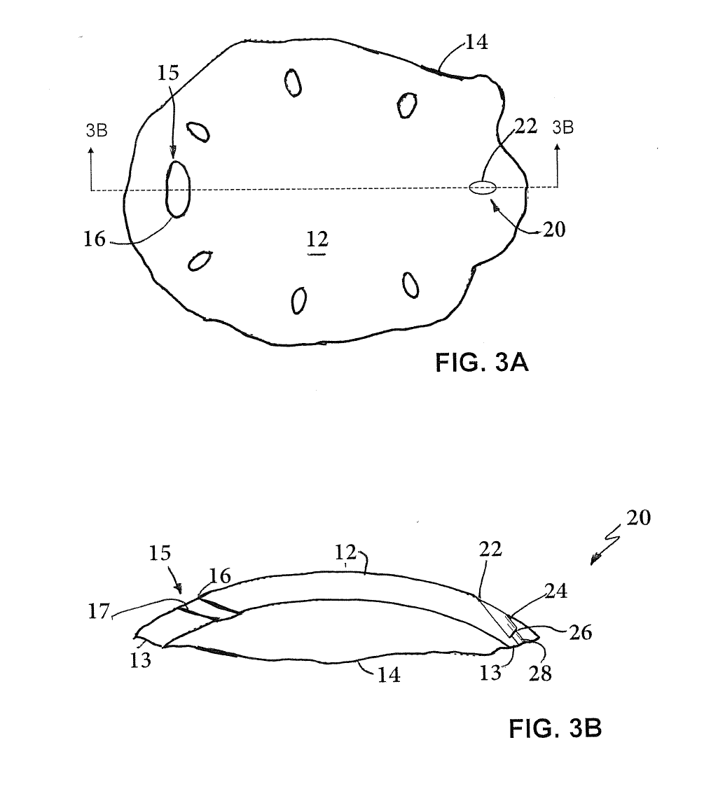

Referring to FIGS. 2, 3A, and 3B, an exemplary cranial implant 10 adapted to replace bone missing, e.g., a bone flap, from a cranial vault is shown. In some embodiments, the implant 10 includes an interior surface 11 and exterior surface 12 defining a shell contour variable thickness between the opposing surfaces 11, 12. Although the thickness of a human skull varies and, hence, the thickness of a cranial implant 10 for a discrete patient, typical implant thicknesses may range between about 4 mm and 10 mm. Those of ordinary skill in the art can appreciate that, although the range provided is typical, actual thicknesses may be greater than or less than the range. A continuous, peripheral surface 13 around a peripheral edge 14 of the implant 10 is formed to mesh with, or marry, the contour of defect edges of the cranium. Those of ordinary skill in the art can appreciate that scan data from a CT scan of the defect edge of the cranium may simplify manufacture of the peripheral edge 14 and the peripheral surface 13 of the edge 14 of the cranial implant 10. More specifically, CT scan data may provide surface features, thicknesses, and other dimensions of the cranial vault area, especially details about the defect edges of the cranial vault, that may be used to design and to manufacture the contour of the implant shell, the peripheral edge 14 of the implant 10, the peripheral surface 13 of the edge 14, and so forth to provide a custom fit with the cranial vault.

In some implementations, at least one drainage channel 15 (or “drain port”) may be formed through the thickness of the implant 10. The drainage channel 15 is configured to allow passage of a removable drain or other conduit (e.g., a hose) between the cranial cavity and the exterior surface 12 of the cranial implant 10. In some variations, the drainage channel 15 is defined by a peripheral opening 16 on the exterior surface 12 of the implant 10, as well an inner channel surface 17. The shape of the peripheral opening 16 may be amorphous, round, oval, elliptical, and the like. In some variations, the obliquity of the drainage channel 15 through the skull of the patient may be such that there is minimal angulation at both the interior surface 11 and the exterior surface 12 of the implant 10, thereby facilitating ease of placement and removal of the drain or other conduit. Typical drainage channel 15 dimensions for the peripheral opening 16 may range between about 5 mm and about 12 mm. Typical angles of obliquity of the drainage channel 15 may range between about 5 degrees and about 20 degrees as measured from a line tangent to the exterior surface 12 of the implant 10 at the location of the drainage channel 15.

Proximate the peripheral edge 14 of the implant 10, a plurality of oblique openings or fixation channels 20 may be formed (e.g., predrilled) through the implant 10 for the purpose of receiving removable fastening devices (e.g., screws, lag screws, and the like) to fixedly secure the implant 10 to suitable, healthy purchase within the cranium. The number and the center-to-center spacing of the fixation channels 20 on the implant 10, as well as and the setback distance from the peripheral edge 14, will vary. Indeed, scan data from a CT scan may be used in design and manufacture of the implant 10, especially the interface between the peripheral surface 13 of the edge 14 and the defect edges of the cranial vault. Although FIGS. 2, 3A, and 3B show a cranial implant having 7 fixation channels 10, that is done for illustrative purposes only. Typically, three or four fixation channels 20, positioned and oriented so that each side of a hypothetical square or triangle superimposed on the implant 10 may contain a fixation device 20, could suffice. Setback distances from the peripheral edge 14 of the implant 10 to the centroid of the opening of the fixation channel 20 at the exterior surface 12 of the implant 10 may range between about 20 mm and about 30 mm. A gap of between about 1 mm and about 3 mm may result between the peripheral edge 14 of the carinal implant 10 and the skull edge.

More particularly, in some variations, the oblique fixation channels 20 may include an outer opening 22, having a substantially oval or elliptical shape, formed at the exterior surface 12 on the implant 10; an upper or outermost, cylindrical or substantially cylindrical portion 24; a transition, or countersunk, portion 26; and a lower or base, cylindrical or substantially cylindrical portion 28. Table I summarizes typical maxima and minima dimensions for the various portions of the fixation channels 20.

TABLE I

| | Min. | Max. |
|---|---|---|
| | (mm) | (mm) |
| Dimensions of | 6 | 12 |
| outer opening | | |
| Length of upper | 5 | 8 |
| portion | | |
| Diameter of | 5 | 10 |
| upper portion | | |
| Depth of | 2 | 5 |
| countersink | | |
| Length of lower | 3 | 5 |
| portion | | |
| Diameter of lower | 2 | 3.5 |
| portion | | |

Advantageously, the upper or outermost, cylindrical or substantially cylindrical portion 24, the countersunk portion 26, and the lower or base, cylindrical or substantially cylindrical portion 28 of each fixation channel 20 are dimensioned to accommodate a fixation device 25 and oriented with respect to the defect edges 38 of the cranium 30, such that, when properly installed, portions of the fixation devices 25 at a proximal end (e.g., the head of the screw or lag screw) do not protrude from the upper cylindrical or substantially cylindrical portion 24 of the oblique fixation channel 20, above the exterior surface 12 of the implant 10, while portions of the fixation devices at a distal end (e.g., the anchor) extending or protruding from or through the peripheral surface 13 of the implant 10 into the defect edges 38 of the cranium 30 fixedly attach the implant 10 to suitable purchase in the skull 31. Preferably, the diameters of the upper or outermost portion 24 and the lower or base portion 28 are selected to provide a clearance hole, such that, when centered within the fixation channel 20, the fixation devices 25 do not contact the peripheral walls of the upper or outermost portion 24 or the lower or base portion 28. Indeed, when properly installed, only the underside of the head portion of the fixation device 25 should contact the transition portion 26. Those of ordinary skill in the art can appreciate that the overall length of the fixation devices 25, as well as its anchor length, and the angle of obliquity of each fixation channel 20 may be varied to provide a myriad of implant attaching schemes, depending on the thickness of the cranial implant 10, as well as to take into account the thickness and strength of the skull 31 of the patient. Typical fixation device lengths may range between about 8 mm and about 15 mm, while typical anchor lengths within suitable purchase of the skull 31 may range between about 5 mm and about 10 mm.

Typical angles of obliquity for the fixation channels 20 differ as a function of the thickness of the skull and the purchase of the bone in the skull 31 receiving the anchors of the fixation devices 25. Ideally, fixation devices 25 anchored within the middle half, the middle third, and/or the middle quarter of the skull 31 are desirable. In some variations, typical angles of obliquity may range between about 5 degrees and 20 degrees, as measured from a line tangent to the exterior surface 12 of the cranial implant 10 at the fixation channel location.

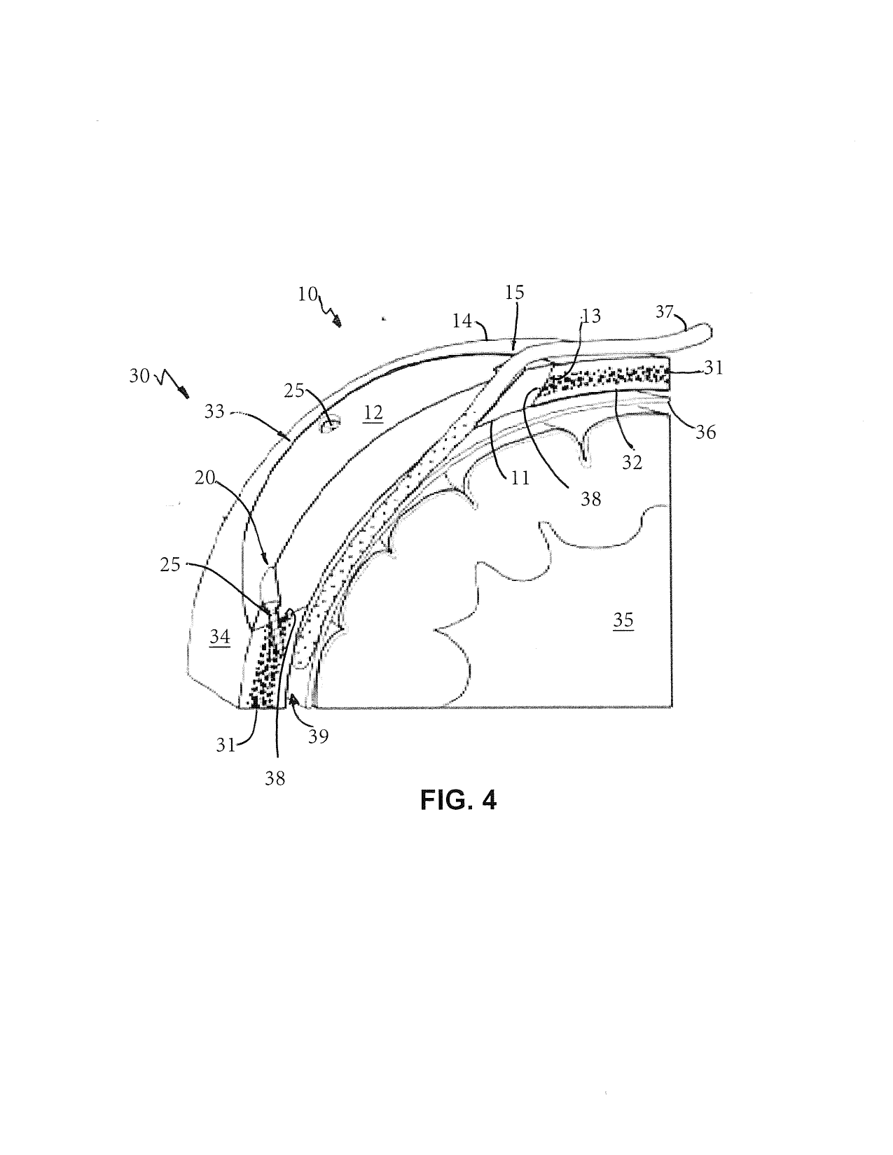

Referring to FIG. 4, an illustrative embodiment of a surgically-emplaced cranial implant 10, in accordance with some aspects of the present invention, is shown. The cranium 30 includes the skull 31 having an inner surface 32 and an outer surface 34, as well as a cranial vault opening 33, e.g., a surgical opening, resulting from removal of a bone flap. Defect edges 38 surround the periphery of the surgical opening 33, which is meant to expose the patient's brain 35 and dura mater 36.

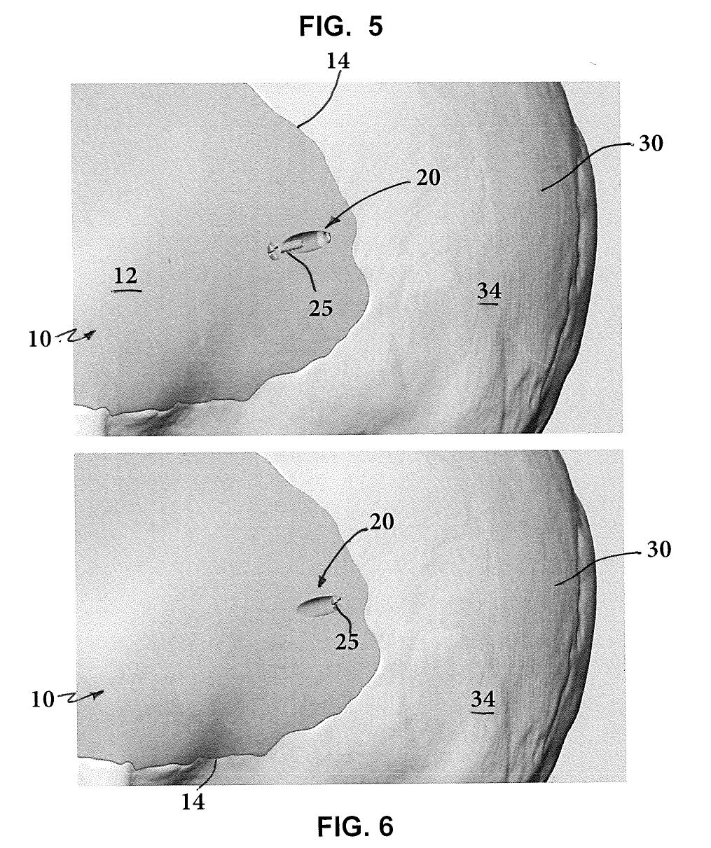

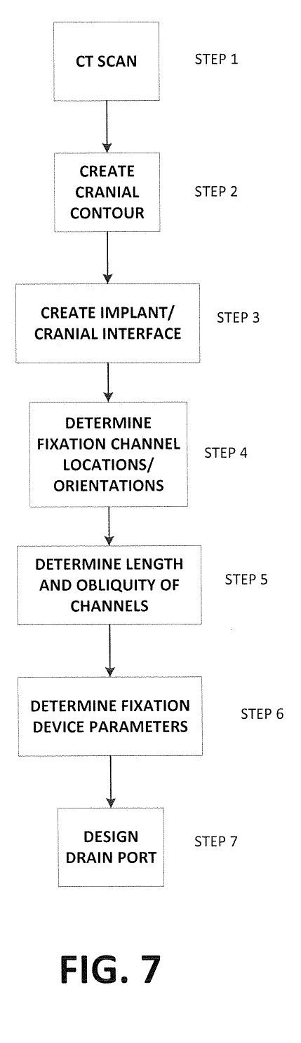

Referring to FIGS. 4-7, in a particular application, the implant 10 may be placed after surgical removal of a portion of the cranium 30, i.e., the bone flap, exposing the cranial defect through appropriate scalp incisions. Typically, soft tissues are removed from defect edges 38 to allow acceptance of the implant 10. Advantageously, the surface 13 of the peripheral edge 14 may be designed, e.g., using three-dimensional data captured during the CT scan, and manufactured to provide an implant/cranium interface structured and arranged, such that the implant 10 may be placed in a unique position determined by the contour of defect edges 38 of the cranium 30, providing a marriage between the defect edges 38 and specific portions of the peripheral surface 13 of the implant 10.

Advantageously, computer-aided design and computer-aided manufacture (CAD/CAM) techniques can be used to custom design and in the manufacture of the implant 10. For example, in a first design/manufacture step, after removal of the bone flap and, optionally, some soft tissue, a high definition, three-dimensional computerized tomography (CT) scan of the patient's cranium 30, including the defect edges 38 of the skull 31, may be obtained (STEP 1). The three-dimensional CT scan (STEP 1) provides CT scan data of the recipient site anatomy necessary for the design of all aspects of the implant 10 and, in particular, the thickness of the skull 31, the condition of the skull 31, the geometry and condition of the defect edges 38 of the cranium 30 surrounding the opening 33, and so forth. In some variations, for example, CT scan data may be used to design the interior surface 11, the exterior surface 12, and the surface 13 of the peripheral edge 14 of the implant 10.

In some implementations, the interior surface 11 and exterior surface 12 of the implant 10 may be designed and manufactured to create an acceptable cranial contour (STEP 2) on both sides of the implant 10, while the surface 13 of the peripheral edge 14 of the implant 10 may be designed and manufactured to form a suitable implant/cranium interface to enhance its mating or marriage with the defect edges 38 (STEP 3) once the implant 10 is properly placed in a unique position. In some variations the cranial contour of the implant 10 may provide a mirror image of the contour of the patient's cranium 30 on the other side of his/her head.

Having designed and manufactured the implant 10 (STEP 2), especially the implant/cranium interface (STEP 3), preferred locations and orientations of fixation channels 20 (STEP 4) proximate the implant/cranium interface, for receiving anchoring fixation devices 25, for securely attaching the implant 10 to the cranium 30, may be determined. Here again, the CT scan data of recipient site anatomy may include thickness and integrity data of the skull 31 about the cranial vault opening 33, from which one of ordinary skill in the art can determine the number, location, and orientation of fixation channels 20 (STEP 4) needed to secure the implant 10 properly. In particular, in some variations, CT scan data may be used to identifying locations of healthy cranial bone suitable for anchoring respective fixation devices 25 in the patient's skull 31, from which information the orientation and location of fixation channels 20 may be determined.

Selection of the location, orientation, obliquity, length, and other dimensions of fixation channels 20, including the various portions of the fixation channel 20, may further be dictated by certain attributes of the cranial bone of the skull 31 gleaned from CT scan data. For example, in some variations, the number and location of the fixation channels 20 about the peripheral edge 14 of the implant 10 may be determined by implant scale and cranial position, e.g., to allow fixation devices 25 to pass obliquely through the implant 10 and purchase healthy cranial bone of the skull 31. Furthermore, a desirable position and orientation of the fixation channels 20 may be selected to avoid penetrating the inner table of the skull to avoid possible brain injury and/or proximity to the sagittal sinus, to avoid the possibility of its inadvertent penetration of the sinus cavity during the cranial hole formation or screw fixation.

Once preferred fixation channel orientations and locations (STEP 4) are determined, the angle of obliquity and dimensions (STEP 5) of each fixation channel 20, as well as the dimensions and type of fixation device 25 (STEP 6), may be determined. Those of ordinary skill in the art can appreciate that design of the location and orientation of the fixation channel 20 (STEP 4), of the dimensions and angle of obliquity of the fixation channel 20 (STEP 5), and of the type and dimensions of the fixation device (STEP 6) may include an iterative process that occurs at the same or substantially the same time. In general, subject to the site anatomy, overall design of the fixation channel 20 and fixation devices 25 prefers a cranial vault 33 of sufficient thickness to allow adequate fixation device anchor length at suitable skull purchase sites for proper anchoring. Thus, CT scan data may be used to determine the shape and/or angle of obliquity of each fixation channel 20; the overall length, anchor length, and other dimensions of the fixation devices 25; and so forth. Typical angles of obliquity and fixation channel lengths, as well as typical fixation device dimensions, are summarized in Table II.

TABLE II

| | Maximum | Minimum |
|---|---|---|
| Angle of Obliquity | 5 | 20 |
| (degrees) | | |
| Fixation Channel | 10 | 18 |
| Length (mm) | | |
| Fixation Device Length | 8 | 15 |
| (Total) (mm) | | |
| Fixation Device Length | 5 | 8 |
| (Anchor) (mm) | | |
| Fixation Device | 1 | 2.5 |
| Diameter (mm) | | |

In some variations, the shape of the fixation channels 20 may be adapted to eliminate or substantially eliminate any fixation hardware 25 protruding from or above the exterior surface 12 of the implant/cranium interface. For example, the shape of each fixation channel 20 may include an upper cylindrical or substantially cylindrical portion 22; a transition, or countersunk, portion 26; and a lower cylindrical or substantially cylindrical portion 28. Those of ordinary skill in the art can appreciate that fixation devices 25 having low-profile head portions may be preferred to those having high-profile head portions. For example, fixation devices 25 with pan, truss, binding, countersunk, and raised-countersunk head portions may be suitable for use.

Indeed, to further the advantage of preventing fixation hardware 25 from protruding from or extending above the exterior surface 12 of the implant 10, in some variations, the upper cylindrical or substantially cylindrical portion 22 may be designed and dimensioned to remove a volume of the implant 10 to enable insertion of the entire fixation device 25, including the threaded anchor and the head portions thereof. The lower cylindrical or substantially cylindrical portion 26 may be designed and dimensioned to remove a volume of the implant 10 to enable insertion of the threaded anchor portion of the fixation device 25 but not the head portion. Finally, the transition, or countersunk, portion 24 may be designed and dimensioned to capture the head portion of the fixation device 25, such that the head portion is capable of exerting a compressive force against the implant 10 to secure the implant 10 to the cranium 30 without any portion of the head portion of the fixation device 25 extending above the exterior surface 12 of the implant 10. Typical maxima and minima dimensions for the various portions of the fixation channels 20 are summarized in Table I.

CT documentation of recipient site anatomy and CT-aided design of the implant 10 may also be used to determine suitable fixation device length (STEP 6), e.g., overall length and anchor length of each fixation device 25. Because the integrity and thickness of the skull 31 may differ about the opening 33, the angle of obliquity of each fixation device location 20 as well as the total length and the anchor length of each fixation device 25 may vary as a function of these varying site anatomy conditions. In some variations, fixation devices 25 (e.g., titanium lag screws) having a 1.5 or 2 mm core diameter may be used. Titanium screws may be preferred as fixation devices 25, because they do not create any artifact during post-operative CT or magnetic resonance imaging.

In some applications, the implant 10 may also be designed to form one or more drainage channels 15, or drain ports, through the implant 10 (STEP 7). In some applications, a drainage channel 15 may be used to provide communication between an intracranial cavity(ies) 39 and the cranial surface 34. More specifically, each drainage channel 15 is located and dimensioned to facilitate evacuating fluids accumulating in any space(s) 39 between an inner surface 32 of the cranium 30 and dura mater 36 and/or brain 35, between the interior surface 11 of the implant 10 and dura mater 36 and/or the brain 35, and/or between dura mater 36 and the brain 35. In some variations, the drainage channel 15 may be dimensioned to allow insertion and passage of a flexible drain line 37, e.g., a conduit, hose, and the like, into the intracranial cavity 39. In some embodiments, the walls of a typical drain line 37 may range in thickness from about 1 mm to about 2 mm. Typical drain lines 37 may have an inner diameter ranging between about 1 mm and about 8 mm and an outer diameter ranging between about 3 mm and about 10 mm. In some variations, dimensions of the drain port 15 may be customized to fit the dimensions of the surgeon's preferred drain line 37. Moreover, drainage channel dimensions may be designed and manufactured to receive a drain line 37 to be included with the implant 10, e.g., with a kit.

After design and manufacture of a custom cranial implant 10, as described above, surgeon's may be provided with the custom cranial implant 10 in a kit that may include a sterilized implant package, e.g., bag, containing the implant 10, as well as a plurality of fixation devices 25 equaling the number of fixation channels 20 formed in the implant 10. Advantageously, the fixation devices 25, e.g., self-drilling screws, provided with the kit may include a length that may be matched to the fixation channel length. In some variations, the kit may further include a drain line 37 for insertion into the drainage channel 15 and/or a device, e.g., a screw driver, Allen wrench, and the like, for installing the fixation devices 25.

While the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements; the present invention being limited only by the claims appended hereto and the equivalents thereof.

What I claim is:

1. A custom cranial implant adapted to be implanted into a cranial opening in a skull of a patient, the cranial implant comprising:
a contoured shell configured to mate with the cranial opening, the contoured shell having a peripheral edge;
a plurality of fixation channels formed within the contoured shell, each fixation channel extending from an exterior surface of the contoured shell into the peripheral edge; and
at least one drain port formed within the contoured shell for receiving at least one of a drain and a conduit.

2. The implant of claim 1, wherein at least one of the fixation channels comprises a countersink feature.

3. The implant of claim 1, wherein each fixation channel is dimensioned to receive a fixation device to attach the contoured shell to the skull of the patient.

4. The implant of claim 3, wherein a head portion of each fixation device is contained within a countersink feature of a corresponding fixation channel, such that the head portion does not protrude above the exterior surface of the contoured shell.

5. The implant of claim 1, wherein the peripheral edge comprises a surface that is configured to mate with defect edges of the cranial opening.

6. A method of manufacturing a custom cranial implant for use in a cranial opening in a skull of a patient, using a three-dimensional image of the cranial opening and surrounding tissue, the method comprising:
forming an implant/skull interface about a peripheral edge of the cranial implant;
determining fixation device locations proximate the peripheral edge of the implant;
forming an oblique channel at each fixation device location, each oblique channel extending from an exterior surface of the contoured shell into the peripheral edge; and
forming at least one drain port through the contoured shell of the cranial implant.

7. The method of claim 6 further comprising forming a cranial contour on the exterior surface of the implant.

8. The method of claim 6, wherein the oblique channels are adapted to allow corresponding fixation devices to pass obliquely through the implant into cranial bone in the skull.

9. The method of claim 6, wherein the oblique channels are adapted to allow corresponding fixation devices to purchase cranial bone of the skull.

10. The method of claim 6 further comprising forming a countersink feature in each oblique channel.

11. The method of claim 10, wherein each countersink feature is configured to contain a head portion of a fixation device, such that the head portion does not protrude above the exterior surface of the cranial implant.

12. The method of claim 6, wherein forming the implant/skull interface comprises forming, using the three-dimensional image, the implant/skull interface about the peripheral edge of the cranial implant to mate with defect edges of the cranial opening.

13. The method of claim 6 further comprising determining at least one fixation device dimension for securing the fixation device to the skull.

14. A method of surgically implanting a custom cranial implant having a contoured shell and a computer designed peripheral edge into a cranial opening in a skull of a patient, the method comprising:
providing a custom cranial implant having at least one drain port;
mating the computer designed peripheral edge to corresponding defect edges of the cranial opening; and
attaching the cranial implant to the skull of the patient.

15. The method of claim 14, wherein providing a custom cranial implant comprises:
using data from a three-dimensional image of the cranial opening to from the custom cranial implant;
using data from a three-dimensional image of the cranial opening to locate a plurality of fixation device locations proximate the peripheral edge of the implant;
forming an oblique fixation channel at each fixation device location, such that each oblique fixation channel extends from an exterior surface of the contoured shell into the peripheral edge; and
forming a drain port extending from the exterior surface of the contoured shell to an interior surface of the contoured shell.

16. The method of claim 15, further comprising:
forming a countersink feature in each oblique fixation channel, each countersink feature configured to contain a head portion of a fixation device, such that the head portion does not protrude above the exterior surface of the contoured shell.

17. The method of claim 14, wherein attaching the cranial implant comprises:
inserting computer designed fixation devices into corresponding oblique fixation channels.

18. The method of claim 17, wherein each fixation device is inserted, such that each fixation devices passes obliquely through the implant into cranial bone in the skull.

19. The method of claim 17, wherein each fixation device is inserted, such that each fixation device purchases cranial bone of the skull of the patient.

20. The method of claim 14 further comprising inserting at least one of a drain and a conduit into the drain port.

21. A kit for implanting an object into a cranial opening in a skull of a patient, the kit comprising:
a custom cranial implant comprising:
a contoured shell configured to mate with the cranial opening and having a peripheral edge,
a plurality of fixation channels formed within the contoured shell, each fixation channel extending from an exterior surface of the contoured shell into the peripheral edge, and
at least one drain port formed within the contoured shell; and
at least one of a drain and a conduit.

22. The kit of claim 21 further comprising a plurality of fixation devices.
